(19) United States
(12) Plant Patent Application Publication
(10) Pub. No.: US 2012/0090063 P1
(43) Pub. Date: Apr. 12, 2012

(54) Stevia plant named ' AKH L4 '

(50) ;

(75) Inventor: Edgar Ramon ALVAREZ Britos, Asuncion (PY)

(73) Assignee: PureCircle Sdn Bhd (578803-K), Negeri Sembilan (MY)

(21) Appl. No.: 13/068,858

(22) Filed: Jan. 18, 2011

Related U.S. Application Data
(60) Provisional application No. 61/336,505, filed on Jan. 22, 2010.

(51) Int. Cl. A01H 5/00

(52) U.S. Cl. PLT/263.1

(57) ABSTRACT

A new and distinct cultivar of stevia plant named ‘AKH L4’, characterized by its combination of early cycle, dark green leaves, medium number of nodes on the main stem, high number of basal buds, high Rebaudioside A of total Glycoside Steviol content, and high yielding of dried leaves at harvest.

### CROSS-REFERENCE TO RELATED APPLICATIONS

Provisional application No. 61/336,505, filed on Jan. 22, 2010.

### STATEMENT REGARDING FEDERALLY SPONSORED RESEARCH OR DEVELOPMENT

None

### GENUS AND SPECIES

Stevia rebaudiana (Bert.) Bertoni

### VARIETY DENOMINATION

‘AKH L4’

### BACKGROUND OF THE INVENTION

The present invention relates to a new and distinct variety of stevia (Stevia rebaudiana (Bert.) Bertoni), which has been given the variety denomination of ‘AKH L4’.

The new stevia plant cultivar is a selection resulting from a controlled breeding program of stevia plants at Guyayybi, San Pedro, Paraguay in 2006 through from the controlled pollination of seed parent “AKH/G.8.D.” (unpatented) and pollen parent “EIRETE” (unpatented). The new cultivar was selected as a single plant within a population of plants resulting from this controlled pollination of stevia plants in 2006 at an experimental station at Guyayvi, San Pedro, Paraguay. Selection criteria was a combination of Rebaudioside A content equal to more than 50% of total steviol glycols, high yield of leaves per hectare, high capacity of sprouting after each harvest and resistance to leaf spot diseases. The selection was subsequently evaluated for a number of years at the Experimental Station of Pure Circle South America at Guyayvi, San Pedro, Paraguay.

Asexual reproduction of the new cultivar by clonal propagation has been used to create mother plants, and then by clonal propagation by cuttings in field nurseries to produce seedlings since 2007 at Guyayvi, San Pedro, Paraguay and has demonstrated that the new cultivar reproduces true to type with all of the characteristics, as herein described, firmly fixed and retained through successive generations of such asexual propagation.

The new variety can be compared to the seed parent “AKH/G.8.D.” In Guyayvi, San Pedro, Paraguay the leaf shape of ‘AKH L4’ is ‘ovo-lanceolate’ whereas the leaf shape of ‘AKH/G.8.D.’ is ‘oval’. The percentage of Stevioside of ‘AKH L4’ is ‘1.3%’ whereas the percentage of Stevioside of ‘AKH/G.8.D.’ is ‘18%’. The percentage of Rebaudioside A of ‘AKH L4’ is ‘11.5%’ whereas the percentage of Rebaudioside A of ‘AKH/G.8.D.’ is ‘0%’.

### SUMMARY OF THE INVENTION

The following characteristics of the new cultivar have been repeatedly observed and can be used to distinguish ‘AKH L4’ as a new and distinct cultivar of a stevia plant:

1. early cycle

2. dark green leaf

3. medium number of nodes on main stem

4. many basal buds

5. Stevioside content 1.3%

6. Rebaudioside A content 11.5%

7. total of Estevioside+Rebaudioside A 12.8%

8. 89% of Rebaudioside A of total Glycoside Steviol

9. yields 4,500 kg/ha of dried leaves in three cuts

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs differ slightly from the color values cited in the detailed description, which accurately describes the colors of ‘AKH L4’.

FIG. 1 illustrates an ‘AKH L4’ plant from roots to top of the plant.

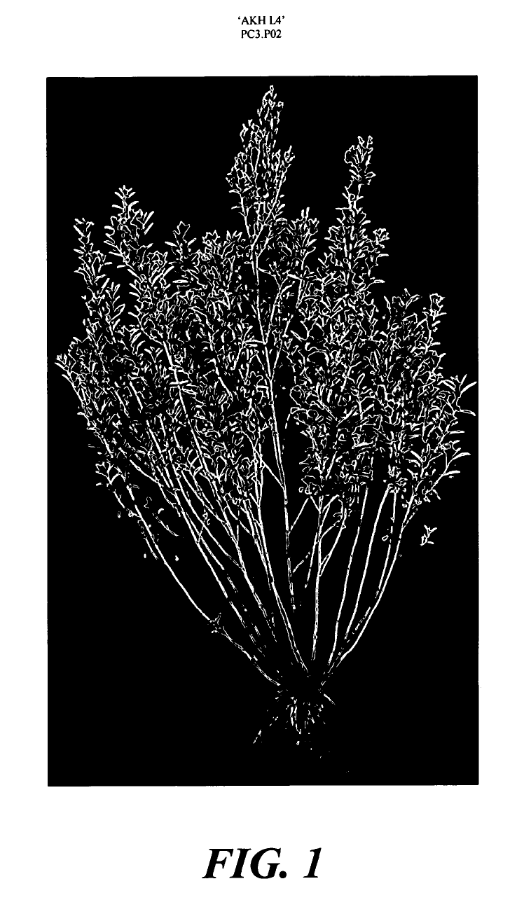

FIG. 2 illustrates ‘AKH L4’ leaves.

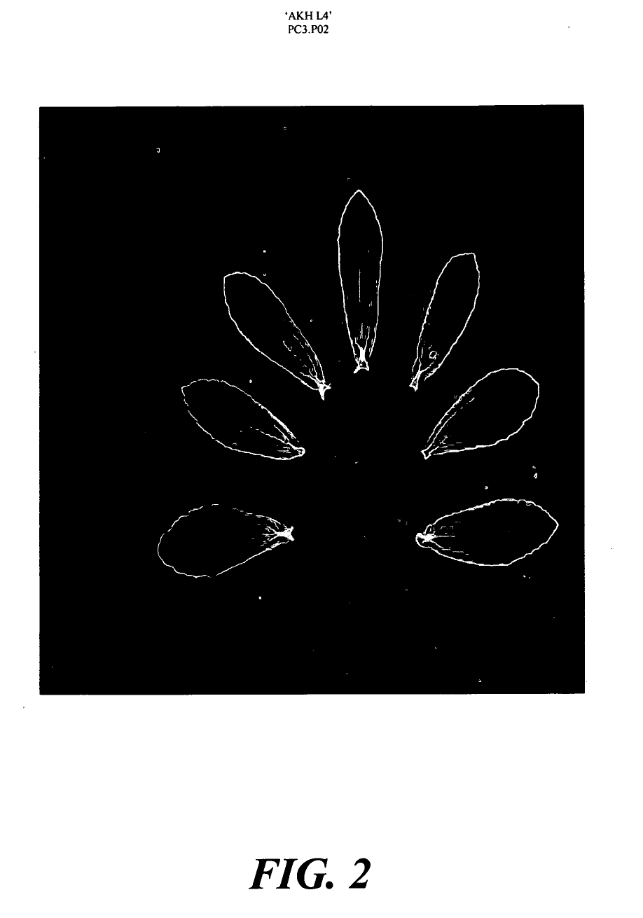

FIG. 3 illustrates an ‘AKH L4’ leaf, including the shape and dimensions of a typical leaf.

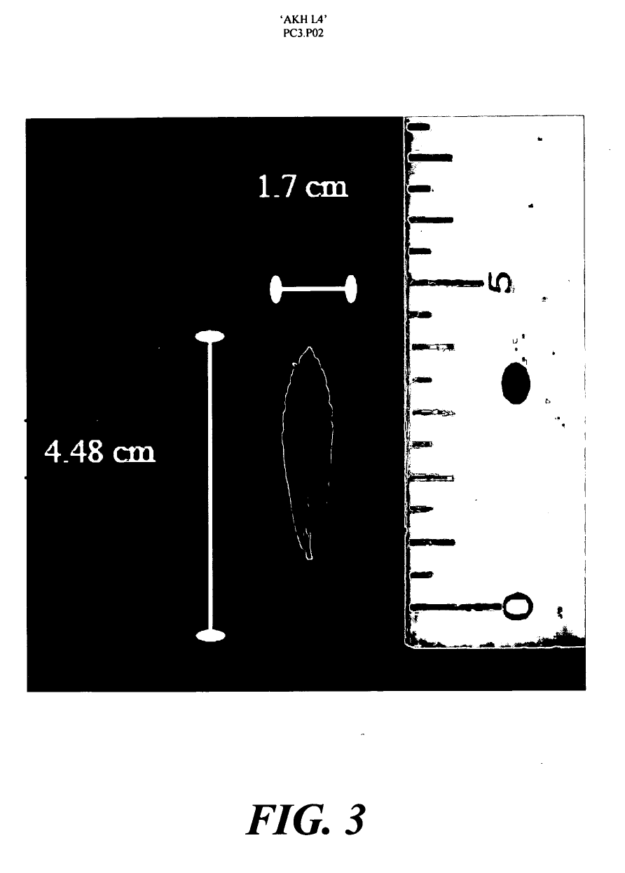

FIG. 4 illustrates the branches, without leaves, of an ‘AKH L4’ plant.

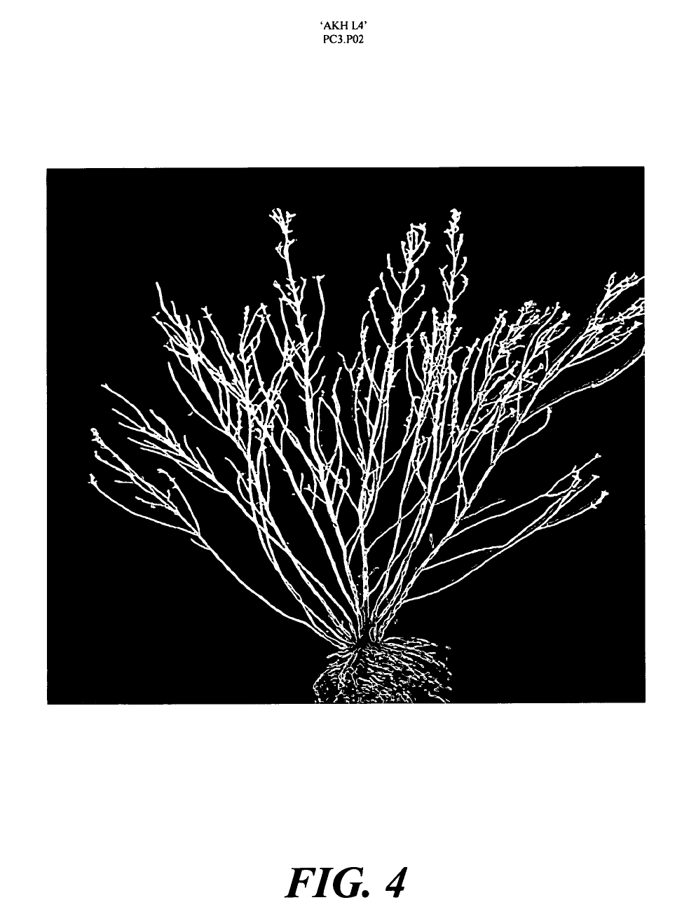

FIG. 5 illustrates the flowers of an ‘AKH L4’ plant.

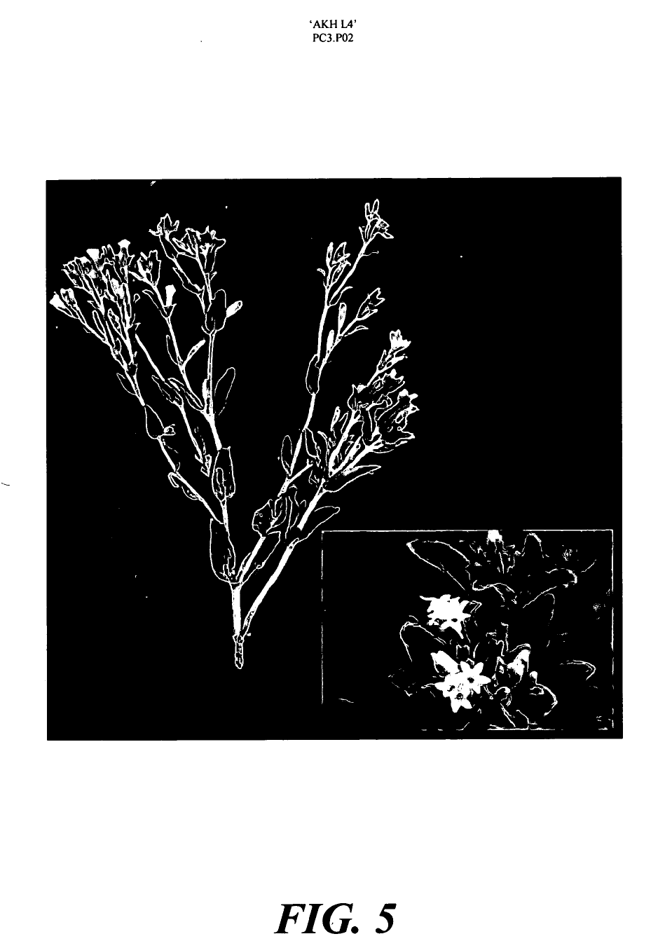

### DETAILED BOTANICAL DESCRIPTION

The following is a detailed botanical description of a new and distinct variety of a stevia plant known as ‘AKH L4’. Plant observations were made on plants grown in Guyayvi, San Pedro, Paraguay. Unless indicated otherwise, the descriptions disclosed herein are based upon observations made in February 2008 of mature ‘AKH L4’ plants (age 6 months) grown in outdoor field plantings with day temperature ranging from 25° C. to 35° C., night temperatures ranging from 22° C. to 30° C., and light levels ranging from 20 to 25 klux. ‘AKH L4’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light quality, light intensity, day length, cultural conditions and the like. Color notations are based on The Royal Horticultural Society Colour Chart, of The Royal Horticultural Society, London, 2007 edition.
Botanical classification: Stevia rebaudiana (Bert.) Bertoni cultivar AKH L4.
Parentage:
   Parents.—seed parent “AKH/G.8.D.” (unpatented); pollen parent “EIRETE” (unpatented).
Plant:
   Growth habit.—prostrate Type III; Main stem with dominance of auxiliary buds and a good development of lateral branches in the auxiliary buds.
   Plant height.—low; approximately 50 cm to 55 cm.
   Number of nodes on main stem.—medium; between 21 to 30 nodes.
   Number of basal buds.—many; 11 to 15 basal buds per plant.
   Cycle.—early.
Fully developed leaf:
   Medium size.—Length 4.48 cm; Width 1.7 cm.
   Shape.—ovo-lanceolate.
   Color.—dark green.
   Leaf edge.—moderately sawn.
   Stevioside content.—3.6%.
   Rebaudioside a content.—9.9%.
   Rebaudioside a of total glycoside steviol.—73%.
   Total of stevioside and rebaudioside a.—13.5%.
   Productivity.—Yielding of dried leaves in three cuts 5,5000 kg/ha to 6,000 kg/ha.
   Cold hardiness.—Cold tolerance from 0° C.
   Heat tolerance.—Heat tolerance to 45° C.
   Pathogen resistance.—Moderately resistant to Septoria steviae.
   Leaf harvest period.—approximately 4 harvests per year; one in each of the following months in Guyayvi, San Pedro, Paraguay: August, November, February, and April.

TABLE 1
Comparison with parents

| Organ/Plant Part: | | | |
|---|---|---|---|
| Context | AKH L4 | AKH/G.8.D. | Eirete |
| Plant: height | low 50 cm-55 cm | low 50 cm-55 cm | high 80 cm |
| Leaf: color | dark green | dark green | dark green |
| Leaf: shape | ovo-lanceolate | oval | lanceolate |
| Leaf: edge | moderately | scalloped | strongly sawn |
| | sawn | | or notched |
| Stevioside content: | 1.3% | 18% | 10.4% |
| Rebaudioside A | 11.5% | 0% | 11.3% |
| content: | | | |
| Number of basal | many 11-15 | many 11-15 | medium 6-10 |
| buds | buds per plant | buds per plant | buds per plant |

What is claimed is:

1. A new and distinct cultivar of stevia plant named ‘AKH L4’, substantially as herein shown and described.
